(19) United States
(12) Patent Application Publication
(10) Pub. No.: US 2023/0141002 A1
(43) Pub. Date: May 11, 2023

(54) DEVICE FOR APPLYING FLUID TO LEAFLETS OF A HEART VALVE PROSTHESIS DURING CRIMPING

(71) Applicant: Medtronic, Inc., Minneapolis, MN (US)

(72) Inventors: Matthew ZIEBOL, Blaine, MN (US); Alkindi KIBRIA, Irvine, CA (US); Victoria UNG, Santa Rosa, CA (US); Matthew GENOVESE, Windsor, CA (US); Yogesh DAREKAR, Irvine, CA (US); Matthew BALDWIN, Santa Rosa, CA (US); Jason BOWE, Blaine, MN (US); Radhika BHARGAV, Mountain View, CA (US)

(21) Appl. No.: 17/967,764

(22) Filed: Oct. 17, 2022

Related U.S. Application Data
(60) Provisional application No. 63/277,788, filed on Nov. 10, 2021.

(51) Int. Cl. A61F 2/24; A61F 2/95

(52) U.S. Cl. CPC A61F 2/2433; A61F 2/2418; A61F 2/9524; A61F 2250/0069

(57) ABSTRACT

A leaflet folding accessory or tool configured for use with a crimper when radially compressing a transcatheter valve prosthesis into a crimped configuration for delivery within a vasculature. The crimping accessory is configured to apply or remove fluid to or from the at least one leaflet of the transcatheter valve prosthesis during the crimping process to prevent protrusion of the leaflets into the frame of the transcatheter valve prosthesis that may cause leaflet pinching and damage.

### CROSS-REFERENCE TO RELATED APPLICATIONS

This application claims the benefit of U.S. Provisional Application No. 63/277,788, filed Nov. 10, 2021, the entire content of which is incorporated herein by reference.

### FIELD OF THE INVENTION

The present invention relates to a crimping accessory for use with a crimper to manage the folding of leaflets of a transcatheter heart valve prosthesis during the crimping process.

### BACKGROUND

The human heart is a four chambered, muscular organ that provides blood circulation through the body during a cardiac cycle. The four main chambers include the right atrium and right ventricle which supplies the pulmonary circulation, and the left atrium and left ventricle which supplies oxygenated blood received from the lungs into systemic circulation. To ensure that blood flows in one direction through the heart, atrioventricular valves (tricuspid and mitral valves) are present between the junctions of the atrium and the ventricles, and semi-lunar valves (pulmonary valve and aortic valve) govern the exits of the ventricles leading to the lungs and the rest of the body. These valves contain leaflets or cusps that open and shut in response to blood pressure changes caused by the contraction and relaxation of the heart chambers. The valve leaflets move apart from each other to open and allow blood to flow downstream of the valve, and coapt to close and prevent backflow or regurgitation in an upstream manner.

Diseases associated with heart valves, such as those caused by damage or a defect, can include stenosis and valvular insufficiency or regurgitation. For example, valvular stenosis causes the valve to become narrowed and hardened which can prevent blood flow to a downstream heart chamber from occurring at the proper flow rate and may cause the heart to work harder to pump the blood through the diseased valve. Valvular insufficiency or regurgitation occurs when the valve does not close completely, allowing blood to flow backwards, thereby causing the heart to be less efficient. A diseased or damaged valve, which can be congenital, age-related, drug-induced, or in some instances, caused by infection, can result in an enlarged, thickened heart that loses elasticity and efficiency. Some symptoms of heart valve diseases can include weakness, shortness of breath, dizziness, fainting, palpitations, anemia and edema, and blood clots which can increase the likelihood of stroke or pulmonary embolism. Symptoms can often be severe enough to be debilitating and/or life threatening.

Heart valve prostheses have been developed for repair and replacement of diseased and/or damaged heart valves. Such heart valve prostheses can be percutaneously delivered and deployed at the site of the diseased heart valve through catheter-based delivery systems. Such heart valve prostheses are delivered in a radially compressed or crimped configuration so that the heart valve prosthesis can be advanced through the patient’s vasculature. Once positioned at the treatment site, the heart valve prosthesis is expanded to engage tissue at the diseased heart valve region to, for instance, hold the heart valve prosthesis in position.

The present disclosure relates to improvements in radially compressing or crimping a heart valve prosthesis to ensure that the leaflets of the heart valve prosthesis are not pinched or damaged during the crimping process.

### BRIEF SUMMARY OF THE INVENTION

In accordance with an aspect hereof, an assembly comprising a transcatheter heart valve prosthesis and a crimping accessory is disclosed. The crimping accessory includes a body and at least one nozzle, the crimping accessory being configured to apply or remove pressurized fluid via the at least one nozzle to or from the at least one leaflet of the transcatheter heart valve prosthesis to hold the at least one leaflet in a substantially closed state when the crimper is radially compressing the transcatheter heart valve prosthesis into the crimped configuration.

In one aspect, the present disclosure provides an assembly comprising a transcatheter heart valve prosthesis including a frame and a valve component including at least one leaflet disposed within and secured to the frame, the transcatheter heart valve prosthesis having a crimped configuration for delivery within a vasculature and an expanded configuration for deployment within a native heart valve, and a crimping accessory configured for use with a crimper when radially compressing the transcatheter heart valve prosthesis into the crimped configuration, wherein the crimping accessory includes a body having an aperture formed on an outer surface thereof, at least one nozzle coupled to the body, and at least one fluid chamber formed between the aperture and the nozzle such that the aperture and the at least one nozzle are in fluid communication, the crimping accessory being configured to apply or remove pressurized fluid via the at least one nozzle to or from the at least one leaflet of the transcatheter heart valve prosthesis to hold the at least one leaflet in a substantially closed state when the crimper is radially compressing the transcatheter heart valve prosthesis into the crimped configuration.

In another aspect hereof, and in combination with any other aspects, the disclosure provides the crimping accessory includes a first end and a second end opposing the first end, the first end and the second end of the crimping accessory being substantially circular in shape.

In another aspect hereof, and in combination with any other aspects, the disclosure provides the first end of the crimping accessory has a first circumference which is larger than a second circumference of the second end of the crimping accessory.

In another aspect hereof, and in combination with any other aspects, the disclosure provides the aperture disposed on the outer surface of the body is configured to receive tubing configured to connect the crimping accessory to a fluid source or a suction source.

In another aspect hereof, and in combination with any other aspects, the disclosure provides the body of the crimping accessory further includes a channel extending from a first end to a second end thereof, the channel being configured to receive a balloon catheter of a delivery system for the transcatheter heart valve prosthesis.

In another aspect hereof, and in combination with any other aspects, the disclosure provides the body of the crimping accessory further includes at least one hinge such that the crimping accessory has a clam-shell configuration that allows the crimping accessory to snap onto the balloon catheter of the delivery system.

In another aspect hereof, and in combination with any other aspects, the disclosure provides the fluid chamber is annular and is disposed radially outward form the channel configured to receive the balloon catheter.

In another aspect hereof, and in combination with any other aspects, the disclosure provides the transcatheter heart valve prosthesis includes exactly three leaflets and the crimping accessory includes exactly three nozzles, each nozzle being configured to remove or apply fluid to or from a leaflet of the exactly three leaflets.

In another aspect hereof, and in combination with any other aspects, the disclosure provides the body of the crimping accessory includes a slot extending from a first end to a second end thereof, the slot being configured to receive a balloon catheter of a delivery system for the transcatheter heart valve prosthesis, and wherein the crimping accessory slides onto the balloon catheter of the delivery system via the slot.

In another aspect hereof, and in combination with any other aspects, the disclosure provides the slot configured to house the balloon catheter extends an entire longitudinal length of the crimping accessory and has a substantially U-shaped cross-section, and wherein the fluid chamber is C-shaped and disposed radially outward from the slot configured to house the balloon catheter.

In another aspect hereof, and in combination with any other aspects, the disclosure provides the crimping accessory is configured to be disposed within and attached to an interior surface of the crimper.

In another aspect hereof, and in combination with any other aspects, the disclosure provides the crimping accessory contains a singular, annular nozzle.

In another aspect hereof, and in combination with any other aspects, the disclosure provides the singular, annular nozzle applies pressurized fluid to the at least one leaflet of the transcatheter heart valve prosthesis.

In another aspect hereof, and in combination with any other aspects, the disclosure provides the singular, annular nozzle removes pressurized fluid from the at least one leaflet of the transcatheter heart valve prosthesis.

In another aspect hereof, and in combination with any other aspects, the disclosure provides the crimping accessory includes a seal around an outer perimeter of the first end configured to seal around the crimper.

In one aspect hereof, and in combination with any other aspects, the present disclosure provides a method of crimping a transcatheter heart valve prosthesis onto a delivery system comprising positioning a transcatheter heart valve prosthesis into a crimper chamber of a crimper, the transcatheter heart valve prosthesis including a frame and a valve component including at least one leaflet disposed within and secured to the frame, wherein the transcatheter heart valve prosthesis is in the expanded configuration, operating the crimper to radially compress the transcatheter heart valve prosthesis into a crimped configuration for delivery within a vasculature, wherein a crimping accessory applies or removes pressurized fluid to or from the at least one leaflet of the transcatheter heart valve prosthesis to hold the at least one leaflet in a substantially closed state during the step of operating the crimper.

In another aspect hereof, and in combination with any other aspects, the disclosure provides the transcatheter heart valve prosthesis may be partially compressed or fully compressed during the operating step.

In another aspect hereof, and in combination with any other aspects, the disclosure provides the crimping accessory includes a body, at least one nozzle and at least one fluid chamber fluidly connecting the at least one nozzle to an aperture disposed on an outer surface of the body, the at least one nozzle being configured to apply or remove pressurized fluid to or from the at least one leaflet of the transcatheter heart valve prosthesis to hold the at least one leaflet in a substantially closed state when the crimper is radially compressing the transcatheter heart valve prosthesis into the crimped configuration.

In another aspect hereof, and in combination with any other aspects, the disclosure provides the crimping accessory is positioned outside the crimper.

In another aspect hereof, and in combination with any other aspects, the disclosure provides the crimping accessory is positioned within the crimper.

In one aspect hereof, and in combination with any other aspects, the present disclosure provides a method of crimping a transcatheter heart valve prosthesis onto a delivery system comprising positioning a transcatheter heart valve prosthesis into a crimper chamber of a crimper, the transcatheter heart valve prosthesis including a frame and a valve component including at least one leaflet disposed within and secured to the frame, wherein the transcatheter heart valve prosthesis is in an expanded configuration and is mounted over a balloon of a balloon catheter, operating the crimper to radially compress the transcatheter heart valve prosthesis on the balloon of the balloon catheter into a partially crimped configuration, removing the partially crimped transcatheter heart valve prosthesis on the balloon catheter from the crimper and placing it within a crimping accessory, while disposed within the crimping accessory, applying pressurized fluid to the at least one leaflet of the transcatheter heart valve prosthesis to hold the at least one leaflet in a substantially closed state, removing the partially crimped transcatheter heart valve prosthesis on the balloon catheter from the crimping accessory and placing it back in the crimper chamber of the crimper, operating the crimper to further radially compress the transcatheter heart valve prosthesis into a fully crimped configuration on the balloon catheter configured for delivery within a vasculature, and removing the transcatheter heart valve prosthesis in the crimped configuration on the balloon catheter of the delivery system from the crimper.

In another aspect hereof, and in combination with any other aspects, the disclosure provides the crimping accessory includes a body, at least one nozzle and at least one fluid chamber fluidly connecting the at least one nozzle to an aperture disposed on an outer surface of the body, the at least one nozzle being configured to radially apply pressurized fluid to the at least one leaflet of the transcatheter heart valve prosthesis to hold the at least one leaflet in a substantially closed state when the crimper is radially compressing the transcatheter heart valve prosthesis into the crimped configuration.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

The foregoing and other features and advantages of the present disclosure will be apparent from the following description of embodiments hereof as illustrated in the accompanying drawings. The accompanying drawings, which are incorporated herein and form a part of the specification, further serve to explain the principles of the present disclosure and to enable a person skilled in the pertinent art to make and use the embodiments of the present disclosure. The drawings may not be to scale.

FIG. 1A shows a side view of a transcatheter heart valve prosthesis according to embodiments thereof.

FIG. 1B shows a top view of the transcatheter heart valve prosthesis of FIG. 1A.

FIG. 2A shows a schematic side view of an assembly according to embodiments thereof, the assembly including a crimper, a crimping accessory, and a balloon catheter, wherein the crimping accessory is configured to snap onto the delivery system.

FIG. 2B shows a perspective view of the crimping accessory of FIG. 2A.

FIG. 2C shows a cross-sectional perspective view of the crimping accessory of FIG. 2A.

FIG. 3A shows a front view of a crimping accessory according to another embodiment hereof, wherein the crimping accessory is configured to slide onto the delivery system.

FIG. 3B shows a cross-sectional perspective view of the crimping accessory of FIG. 3A.

FIG. 4A shows a perspective view of a crimping accessory according to another embodiment hereof, wherein the crimping accessory has an annular nozzle configuration.

FIG. 4B shows a cross-sectional perspective view of the crimping accessory of FIG. 4A.

FIG. 5A shows a schematic side view of an assembly according to embodiments thereof, the assembly including a crimper, a plurality of crimping accessories, and a balloon catheter disposed through a crimper chamber of the crimper, wherein the crimping accessories are disposed within an interior of the crimper.

FIG. 5B shows a schematic front view of the interior of the crimper of FIG. 5A.

FIG. 5C shows a perspective side view of a crimping accessory of FIG. 5A, wherein the crimping accessory is removed from the crimper for sake of illustration.

FIG. 6A shows a schematic side view of an assembly according to embodiments thereof, the assembly including a crimper, a crimping accessory, and a balloon catheter, wherein the crimping accessory is configured to be coupled to an outer surface of the crimper.

FIG. 6B shows a perspective view of the crimping accessory of FIG. 6A.

FIG. 6C shows a cross-sectional perspective view of the crimping accessory of FIG. 6A.

FIG. 7A shows a sectional perspective view of a crimping accessory according to another embodiment thereof.

FIG. 7B shows a cross-sectional perspective view of the crimping accessory of FIG. 7A.

FIG. 7C shows a perspective top view of the crimping accessory of FIG. 7A.

FIG. 8 is a block diagram that shows a method of using the crimping accessory of FIGS. 7A-7C according to an embodiment hereof.

### DETAILED DESCRIPTION

It should be understood that various embodiments disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single device or component for purposes of clarity, it should be understood that the techniques of this disclosure may be performed by a combination of devices or components associated with, for example, a medical device. The following detailed description is merely exemplary in nature and is not intended to limit the invention of the application and uses of the invention. Furthermore, there is no intention to be bound by any expressed or implied theory presented in the preceding field of the invention, background, summary or the following detailed description.

As used in this specification, the singular forms “a”, “an” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%. It should be understood that use of the term “about” also includes the specifically recited number of value.

Embodiments hereof relate to a leaflet folding accessory or tool configured for use with a transcatheter valve prosthesis when radially compressing the transcatheter valve prosthesis into a crimped configuration for delivery within a vasculature. More particularly, the crimping accessory is configured to apply or remove fluid to or from the leaflets of a transcatheter heart valve prosthesis during the crimping process to prevent protrusion of the leaflets into the frame of the transcatheter heart valve prosthesis that may cause leaflet pinching and damage. The crimping accessory is spaced a distance from the transcatheter valve prosthesis during the crimping process such that it may apply or remove pressurized fluid to or from the leaflets of the transcatheter heart valve prosthesis without directly contacting the leaflets, thereby eliminating the chance of damage to the leaflets from the crimping accessory.

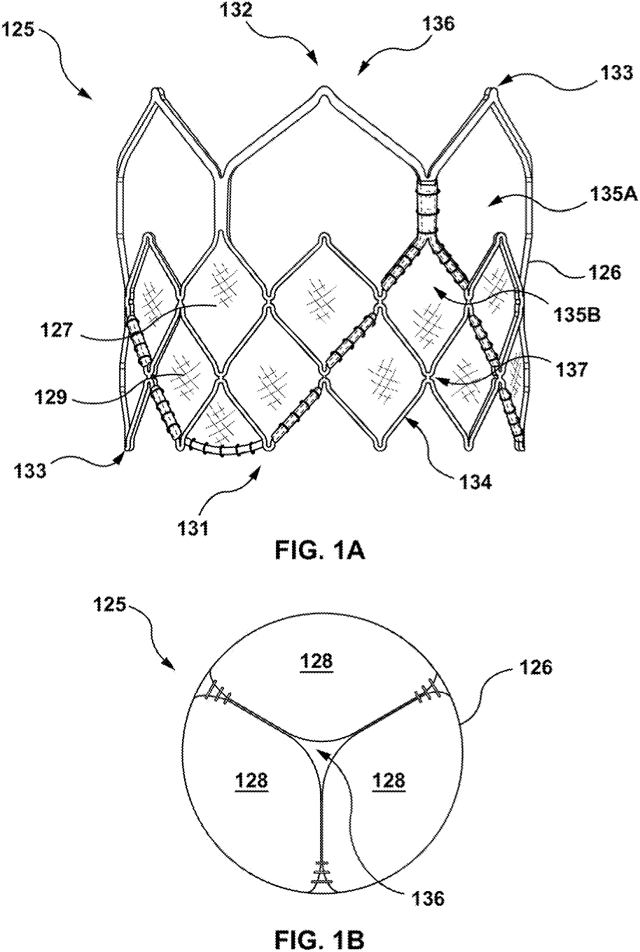

FIGS. 1A and 1B illustrate a transcatheter heart valve prosthesis 125 that may be utilized with the embodiments of the crimping accessory described herein. The transcatheter heart valve prosthesis 125 is illustrated herein in order to facilitate description of the present invention. The following description of the transcatheter heart valve prosthesis 125 is merely exemplary in nature and is not intended to limit the invention or the application and uses of the invention. It is understood that any number of alternate heart valve prostheses can be used with the leaflet accessory tools and methods described herein. Other non-limiting examples of transcatheter heart valve prostheses that can be used with the leaflet accessory tools described herein are described in U.S. Pat. App. No. 17/186,485, filed Feb. 26, 2021, which is incorporated by reference herein in its entirety. Although the transcatheter heart valve prosthesis 125 is a balloon-expandable heart valve prosthesis configured for placement within an aortic heart valve, embodiments of the crimping accessory described herein may be utilized with any transcatheter valve prosthesis that is crimped onto a delivery system. For example, embodiments of the leaflet folding accessory described herein may be utilized with a transcatheter heart valve configured for placement within a pulmonary, aortic, mitral, or tricuspid valve, or may be utilized with a transcatheter valve prosthesis configured for placement within a venous valve or within other body passageways where it is deemed useful. There is no intention to be bound by any expressed or implied theory presented in the preceding technical field, background, brief summary or the following detailed description.

FIGS. 1A and 1B illustrate side and top views, respectively, of the transcatheter heart valve prosthesis 125. The transcatheter heart valve prosthesis 125 includes a radially-expandable frame 126 and a valve component 127. The frame 126 of the transcatheter heart valve prosthesis 125 is a unitary stent or scaffold that supports the valve component 127 within the interior of the frame 126. In an embodiment, the frame 126 is balloon-expandable. The valve component 127 includes at least one leaflet 128 disposed within and secured to the frame 126. In an embodiment, the valve component 127 of the transcatheter heart valve prosthesis 125 includes exactly three leaflets 128, as shown in FIG. 1B. The valve component 127 of the transcatheter heart valve prosthesis 125 is capable of blocking flow in one direction to regulate flow there-through via the valve leaflets 128. The transcatheter heart valve prosthesis 125 has a crimped configuration for delivery within a vasculature and an expanded configuration (as shown in FIGS. 1A and 1B) for deployment within a native heart valve.

As shown in FIG. 1A, the transcatheter heart valve prosthesis 125 includes an inflow portion 131 and an outflow portion 132. The frame 126 of the transcatheter heart valve prosthesis 125 includes a plurality of struts 134 that are arranged to form a plurality of side openings or cells 135 arranged circumferentially around a longitudinal axis of the transcatheter heart valve prosthesis 125 and longitudinally to form a tubular structure defining a central lumen 136 of the transcatheter heart valve prosthesis 125. The struts 134 are defined as the straight segments of the frame 126. Two struts 134 come together to form a crown 133 and four struts 134 come together to form a node 137, as can be seen in FIG. 1A. Each cell 135 of the plurality of cells 135 are disposed within exactly four struts 134 and exactly four nodes 137, or exactly three nodes 137 and exactly one crown 133. The frame 126 is configured to secure the valve component 127 within the central lumen 136 of the frame 126 and to secure the transcatheter heart valve prosthesis 125 in place in the vasculature of the patient.

The inflow portion 131 of the transcatheter heart valve prosthesis 125 includes a plurality of crowns 133 with each crown 133 being formed between a pair of adjacent struts 134. Similarly, the outflow portion 132 of the transcatheter heart valve prosthesis 125 includes a plurality of crowns 133 with each crown 133 being formed between a pair of adjacent struts 134. The frame 126 of the heart valve prosthesis 125 includes a plurality of cells 135 defined as the spaces between the plurality of crowns 133, the plurality of nodes 137 and the plurality of struts 134. In an embodiment, the plurality of cells 135 may be diamond-shaped, as shown in FIG. 1A. In the embodiment described herein, the plurality of cells 135A located at the outflow portion 132 are heart-shaped and are relatively larger than the plurality of cells 135B located at the inflow portion 132 of the frame 126 to improve access to the coronary arteries. More particularly, the cells 135A located at the outflow portion 132 are configured to be of sufficient size to be easily crossed with a coronary guide catheter into either the right coronary artery or the left main coronary artery once the transcatheter heart valve prosthesis 125 is deployed in situ.

The valve component 127 of the transcatheter heart valve prosthesis 125 is capable of regulating flow therethrough via valve leaflets 128 that may form a replacement valve. FIGS. 1A and 1B illustrate an exemplary valve component 127 having three leaflets 128, although a single leaflet or bicuspid leaflet configuration may alternatively be used in embodiments hereof. When deployed in situ, the valve component 127 in a closed state is configured to block blood flow in one direction to regulate blood flow through the central lumen 136 of the frame 126 of the transcatheter heart valve prosthesis 125. FIG. 1A depicts a side view of the transcatheter heart valve prosthesis 125, wherein the valve component 127 is shown disposed within and secured to the frame 126 of the heart valve prosthesis 125. FIG. 1B depicts an atrial or inflow end view of the transcatheter heart valve prosthesis 125 shown in FIG. 1A. The valve component 127 includes valve leaflets 128, e.g., three valve leaflets 128, such that the valve leaflets 128 open during diastole.

Leaflets 128 may be attached to a graft material or skirt 129 which encloses or lines a portion of the frame 126 as would be known to one of ordinary skill in the art of prosthetic tissue valve construction, for example, using sutures or a suitable biocompatible adhesive. Leaflets 128 are sutured or otherwise securely and sealingly attached along their bases to the interior surface of the graft material, or otherwise attached to the frame 126. Adjoining pairs of leaflets are attached to one another at their lateral ends to form commissures, with free edges of the leaflets 128 forming coaptation edges that meet in a closed configuration. The orientation of the leaflets 128 within the valve component 127 depends upon on which end of the transcatheter heart valve prosthesis 125 is the inflow end 131 and which end of the transcatheter heart valve prosthesis 125 is the outflow end 132, thereby ensuring one-way flow of blood through the transcatheter heart valve prosthesis 125.

The valve leaflets 128 and graft material may be formed of various flexible materials including, but not limited to natural pericardial material such as tissue from bovine, equine or porcine origins, or synthetic materials such as polytetrafluoroethylene (PTFE), DACRON® polyester, pyrolytic carbon, or other biocompatible materials. With certain prosthetic leaflet materials, it may be desirable to coat one or both sides of the replacement valve leaflet with a material that will prevent or minimize overgrowth. It is further desirable that the prosthetic leaflet material is durable and not subject to stretching, deforming, or fatigue.

As shown in FIGS. 1A and 1B, the valve component 127 includes at least one leaflet 128 disposed within and secured to the frame 126 of the transcatheter heart valve prosthesis 125. A crimper may be used to radially compress the transcatheter heart valve prosthesis 125 from an expanded configuration to a crimped configuration for delivery within a vasculature. During the crimping process, radial compression exerted by the crimper causes the area of the cells 135 of the frame 126 to decrease. In some instances, the at least one leaflet 128 of the valve component 127 may protrude through the cells 135A at the outflow portion 132 of the frame 126 during the process, which may cause the leaflets 128 to get pinched between struts 134 of the frame 126 and sustain damage. Leaflet protrusion becomes increasingly likely as the area of the cells 135 increase and/or in view of higher crimping forces that are needed to crimp balloon expandable implants.

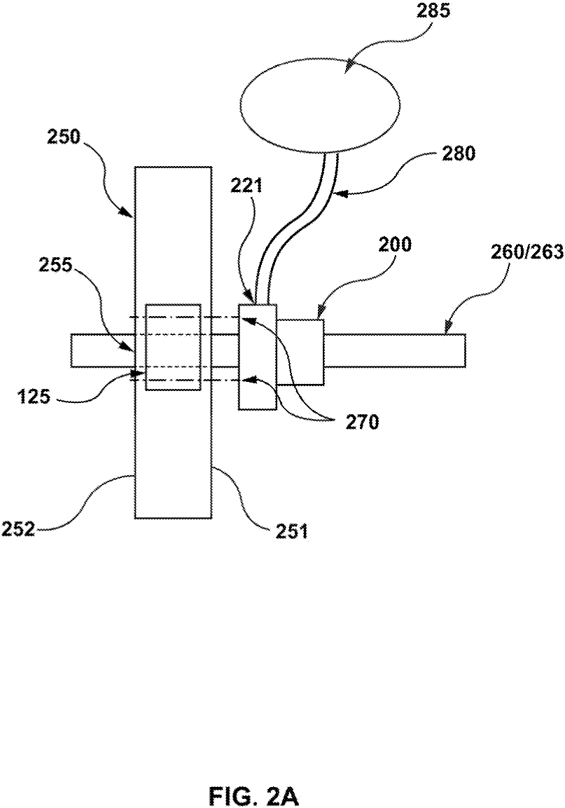

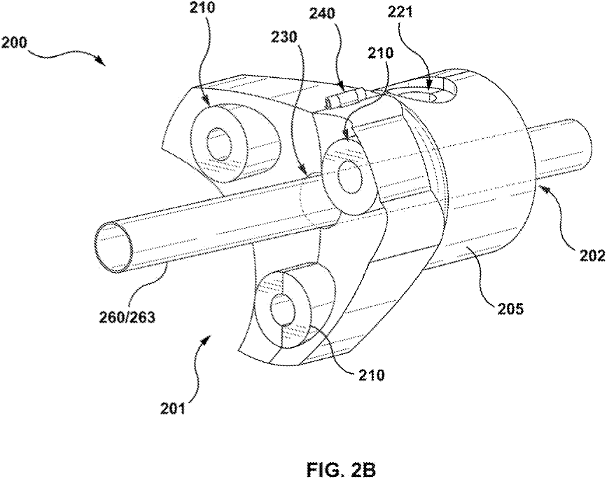

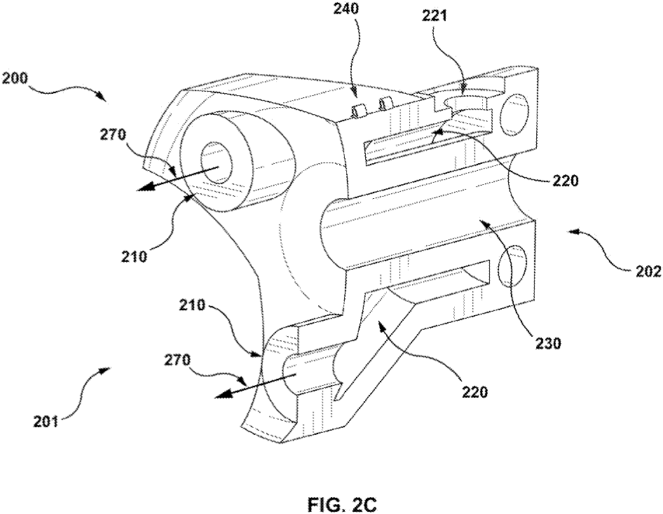

FIG. 2A is a schematic illustration of an assembly including a crimper 250, a crimping accessory 200, and a delivery system 260 including a shaft 263 during the crimping procedure according to embodiments hereof. The crimping accessory 200 is configured for use with a transcatheter valve prosthesis, such as but not limited to the transcatheter heart valve prosthesis 125 described herein, when radially compressing the transcatheter valve prosthesis into a crimped configuration for delivery within a vasculature. For illustrative purposes only, the crimping accessory 200 will be described for use with the transcatheter heart valve prosthesis 125 since the structure thereof has been described herein. One skilled in the art would understand that when using the crimper 250 to compress the transcatheter valve prosthesis 125, the transcatheter heart valve prosthesis 125 is first loaded onto a balloon (not shown) of the delivery system 260 in an expanded configuration. The assembly of the transcatheter heart valve prosthesis 125 and the delivery system 260 is then positioned into a crimper chamber 255 of the crimper 250, as can be seen in FIG. 2A. Prior to the crimping procedure, the crimping accessory 200 is secured onto the shaft 263 of the delivery system 260 and is spaced a distance from a first end 251 of the crimper 250 such that it may apply pressurized fluid to the at least one leaflet 128 of the transcatheter heart valve prosthesis 125 during the crimping process without directly contacting the transcatheter heart valve prosthesis 125. Stated another way, the crimping accessory 200 is configured to interact with the leaflets 128 of the transcatheter heart valve prosthesis 125 in a contactless manner. An aperture 221 of the crimping accessory 200 receives a tubing 280 that connects to a fluid source 285 such that the fluid source 285 may provide pressurized fluid to the crimping accessory 200. The term “fluid” as used herein includes gases (i.e., air) as well as liquids such as water, saline, etc. In an embodiment, the fluid is pressurized with a pressure between 1 PSI and 10 PSI. The crimping accessories described herein are shown and described with a crimper that is oriented in a horizonal orientation for ease of use, and the crimping accessory provides fluid to the transcatheter heart valve prosthesis disposed within the crimper chamber of the crimper in an axial direction. Thus, the crimping accessory is configured to provide the fluid under pressure, i.e., a pressurized fluid, to the at least one leaflet of the transcatheter heart valve prosthesis during the crimping process due to the horizonal orientation of the crimper. However, this is not meant to be limiting and it should be understood that the fluid being applied by the crimping accessory to the at least one leaflet of the transcatheter heart valve prosthesis is not required to be pressurized if the crimper and crimping accessory are oriented in a vertical orientation (i.e., the crimper being disposed below the crimping accessory in a vertical manner) rather than the horizontal orientation shown and described herein.

FIG. 2B shows a crimping accessory 200 according to embodiments herein. The crimping accessory 200 includes a body 205, a first end 201, and a second end 202 that opposes the first end 201. The first end 201 of the crimping accessory 200 has an area larger than an area of the second end 202. The crimping accessory 200 includes a channel 230 configured to receive the shaft 263 of the delivery system 260 as shown in FIG. 2B. The channel 230 extends an entire longitudinal length of the crimping accessory 200, extending from the first end 201 to the second end 202 of the body 205. In an embodiment, the channel 230 has a substantially circular cross-section that is sized slightly greater than an outer diameter of the shaft 263 of the delivery system 260. In embodiments, the body 205 of the crimping accessory 200 may include at least one hinge 240 such that the crimping accessory 200 has a clam-shell configuration. The clam-shell configuration allows the crimping accessory 200 to snap onto the shaft 263 of the delivery system 260 via the channel 230. More particularly, via the hinge 240, the crimping accessory 200 has an open configuration in which the channel 230 is enlarged and the crimping accessory 260 can be disposed over the shaft 263 of the delivery system 260 and a closed configuration in which the channel 230 resumes its substantially circular cross-section and the body 205 of the crimping accessory 200 encloses or surrounds the shaft 263 of the delivery system 260.

The crimping accessory 200 further includes at least one nozzle 210, at least one fluid chamber 220, and an aperture 221, as shown in FIG. 2C. The fluid chamber 220 fluidly connects the at least one nozzle 210 to the aperture 221 disposed on an outer surface of the body 205. In the embodiment shown, the fluid chamber 220 is annular and disposed radially outward from the channel 230 configured to house the shaft 263 of the delivery system 260. The aperture 221 is configured to receive the tubing 280 (shown in FIG. 2A). The tubing 280 is configured to provide fluid from the fluid source 285 to the at least one nozzle 210, as can be seen in FIG. 2A. In the embodiment shown, the body 205 contains exactly three nozzles 210 configured to apply pressurized fluid to the three leaflets 128 of the transcatheter heart valve prosthesis 125. The arrows in FIG. 2C represent a fluid path 270 within the crimping accessory 200 such that the fluid exits through the exactly three nozzles 210 and is directed to and aimed at the three leaflets 128 of the transcatheter heart valve prosthesis 125. Each nozzle 210 is configured to apply fluid to a leaflet 128. Each nozzle 210 is aligned with a center of each leaflet 128 such that when the pressurized fluid is applied, each leaflet 128 is held in a substantially closed state when the crimper 250 is radially compressing the transcatheter heart valve prosthesis 125 into the crimped configuration. In this embodiment, the three nozzles 210 are equally and circumferentially spaced apart from each other, as can be seen in FIG. 2B. The term “substantially closed state” is used herein to describe the leaflets 128 of the valve component 127 being positioned such that the free edges thereof abut against or contact the shaft 263 of the delivery system 260. The closed state of the leaflets 128 against the shaft 263 of the delivery system 260 is similar to the closed state of the leaflets 128 within the vasculature of the patient, in which the leaflets 128 are configured to block blood flow in one direction to regulate the blood flow through the central lumen 136 of the heart valve prosthesis 125, except that the shaft 263 of the delivery system 260 extends through the central lumen 136 of the heart valve prosthesis 125 during the crimping procedure. In another embodiment, when the pressurized fluid is applied, each leaflet 128 is displaced radially inward to prevent leaflet protrusion but does not abut against or contact the shaft 263 of the delivery system when the crimper 250 is radially compressing the transcatheter heart valve prosthesis 125 into the crimped configuration.

A method of using the crimping accessory 200 during the crimping process of the transcatheter heart valve prosthesis 125 will now be described. Referring to FIG. 2A, the transcatheter heart valve prosthesis 125 is first loaded onto the balloon of the balloon catheter of the delivery system 260 in an expanded configuration. The assembly of the transcatheter heart valve prosthesis 125 and the delivery system 260 is then positioned within the crimper chamber 255 of the crimper 250. The crimping accessory 200 is then placed on the shaft 263 of the delivery system 260 and is spaced a distance from the first end 251 of the crimper 250. When the crimper 250 operates to radially compress the transcatheter heart valve prosthesis 125 into a crimped configuration, the crimping accessory 200 applies pressurized fluid to the at least one leaflet 128 of the transcatheter heart valve prosthesis 125 to urge the at least one leaflet 128 into a substantially closed state during the crimping process. FIG. 2A shows the fluid path 270 in phantom for illustrative purposes only from the at least one nozzle 210 to the at least one leaflet 128 of the transcatheter heart valve prosthesis 125 when the prosthesis 125 is being radially compressed within the crimper 250. The fluid from the at least one nozzle 210 is applied axially to an exterior surface of the at least one leaflet 128 of the transcatheter heart valve prosthesis 125 such that the fluid pushes the at least one leaflet 128 radially inwards. Displacing the at least one leaflet 128 radially inwards during the crimping process minimizes the risk of leaflet protrusion and damage. When fluid is applied to the leaflets 128, pooling of the fluid may occur on or around the leaflets 128 of the valve component 127. Such pooling can be beneficial to displacing the leaflets 128 radially inwards. When the transcatheter heart valve prosthesis 125 is fully crimped onto the delivery system 260, the crimping accessory 200 may be removed from the shaft 263 of the delivery system 260 and the assembly of the transcatheter heart valve prosthesis 125 and the delivery system 260 may be removed from the crimper chamber 255 of the crimper 250.

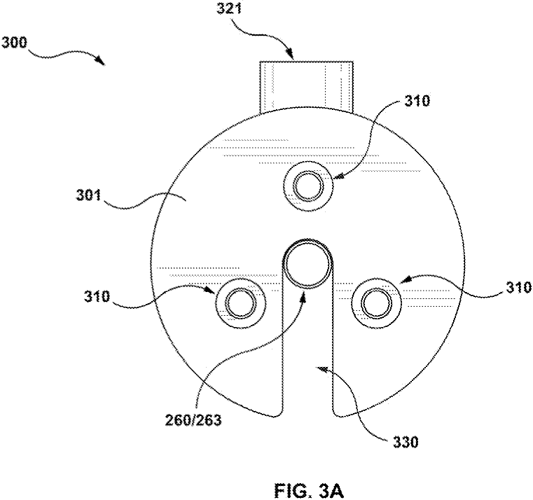

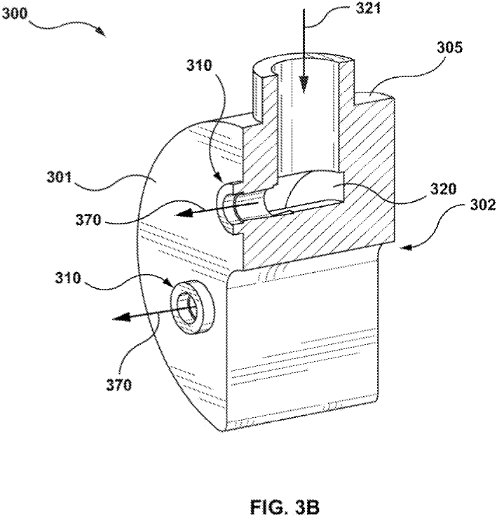

FIGS. 3A-3B show a crimping accessory 300 according to embodiments herein. As compared to the crimping accessory 200 described above, the crimping accessory 300 described herein includes a slot that receives the shaft 263 of the delivery system 260 and allows the crimping accessory 300 to slide onto the delivery system 260. The slot, having a substantially U-shaped cross-section, results in the crimping accessory 300 having a fluid chamber that is substantially C-shaped and disposed radially outward from the slot, as will be described in more detail below.

The crimping accessory 300 described herein includes a body 305, a first end 301, and a second end 302. The first end 301 and the second end 302 of the crimping accessory 300 are substantially circular in shape. The first end 301 has a first circumference larger than the second circumference of the second end 302. The crimping accessory 300 includes a slot 330 configured to receive the shaft 263 of the balloon catheter of the delivery system 260, as shown in FIG. 3A. The slot 330 extends an entire longitudinal length of the crimping accessory 300, extending from the first end 301 to the second end 302 of the body 305, as shown in FIG. 3B. The slot 330 has a substantially U-shaped cross-section that allows the crimping accessory 300 to slide onto the shaft 263 of the balloon catheter of the delivery system 260. Once the transcatheter heart valve prosthesis 125 is loaded onto the balloon of the delivery system 260 and is placed within the crimper chamber 255 of the crimper 250, the crimping accessory 300 slides onto the shaft 263 of the balloon catheter and is spaced a distance away from the first end 251 of the crimper 250 such that it may apply pressurized fluid to the at least one leaflet 128 of the transcatheter heart valve prosthesis 125 during the crimping process without directly contacting the transcatheter heart valve prosthesis 125, similar to the assembly shown in FIG. 2A.

The crimping accessory 300 includes at least one nozzle 310, a fluid chamber 320, and an aperture 321, as shown in FIG. 3B. The fluid chamber 320 fluidly connects the at least one nozzle 310 to the aperture 321 disposed on an outer surface of the body 305. In the embodiment shown, the fluid chamber 320 is C-shaped and is disposed radially outward from the slot 330 configured to house the shaft 263 balloon catheter of the delivery system 260. The aperture 321 is configured to receive the tubing 280. The tubing 280 is configured to provide fluid from the fluid source 285 to the at least one nozzle 310, similar to the assembly shown in FIG. 2A. In the embodiment shown, the body 305 contains three nozzles 310 configured to apply pressurized fluid to the at least one leaflet 128 of the transcatheter heart valve prosthesis 125. The arrows in FIG. 3B represent a fluid path 370 within the crimping accessory 300 such that the fluid exits through the exactly three nozzles 310 and is directed to and aimed at the at least one leaflet 128 of the transcatheter heart valve prosthesis 125. In some embodiments, the transcatheter heart valve prosthesis 125 includes exactly three leaflets 128 and the crimping accessory 300 includes exactly three nozzles 310. In such embodiments, each nozzle 310 is configured to apply fluid to a leaflet 128 of the exactly three leaflets 128. Each nozzle 310 is aligned with a center of each leaflet 128 such that when the pressurized fluid is applied axially, each leaflet 128 is held in a substantially closed state when the crimper 250 is radially compressing the transcatheter heart valve prosthesis 125 into the crimped configuration. In another embodiment, when the pressurized fluid is applied, each leaflet 128 is displaced radially inward to prevent leaflet protrusion but does not abut against or contact the shaft 263 of the delivery system. In the embodiment of FIG. 3A, the three nozzles 310 are equally and circumferentially spaced apart from each other.

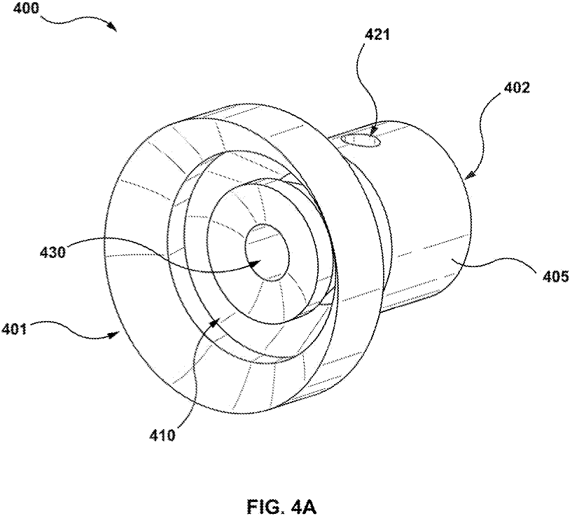

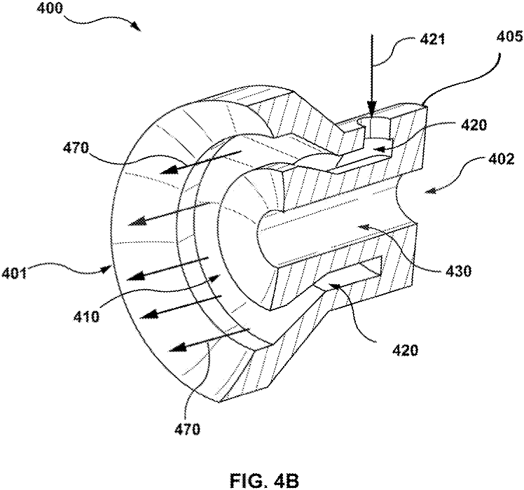

FIGS. 4A-4B show a crimping accessory 400 according to embodiments herein. As compared to the embodiments described above, the crimping accessory 400 described herein contains a singular, annular nozzle configured to apply pressurized fluid to the at least one leaflet 128 of the transcatheter heart valve prosthesis 125 during the crimping process. The crimping accessory 400 described herein includes a body 405, a first end 401, and a second end 402. The first end 401 and the second end 402 of the crimping accessory 400 are substantially circular in shape. The first end 401 has a first circumference larger than a second circumference of the second end 402. The crimping accessory 400 includes a channel 430 configured to receive the shaft 263 of the balloon catheter of the delivery system 260. The channel 430 extends an entire longitudinal length of the crimping accessory 400, extending from the first end 401 to the second end 402 of the body 405. The channel 430 has a substantially circular cross-section.

The crimping accessory 400 includes at least one nozzle 410, a fluid chamber 420, and an aperture 421, as shown in FIG. 4B. The fluid chamber 420 fluidly connects the at least one nozzle 410 to the aperture 421 disposed on an outer surface of the body 405. In the embodiment shown, the fluid chamber 420 is annular and disposed radially outward from the channel 430 configured to house the shaft 263 of the balloon catheter 260 of the delivery system. The aperture 421 is configured to receive the tubing 280. The tubing 280 is configured to provide fluid from the fluid source 285 to the at least one nozzle 410. In the embodiment shown, the body 405 contains a singular, annular nozzle 410 configured to apply pressurized fluid in a substantially annular fluid stream to the at least one leaflet 128 of the transcatheter heart valve prosthesis 125. The arrows in FIG. 4B represent a fluid path 470 within the crimping accessory 400 such that the fluid exits through the annular nozzle 410 and is directed to and aimed at the at least one leaflet 128 of the transcatheter heart valve prosthesis 125. In instances where more fluid may be required to displace the at least one leaflet 128 of the transcatheter heart valve prosthesis 125 radially inward, the crimping accessory 400 having the single annular nozzle 410 is able to apply more fluid and a higher pressure compared to embodiments containing multiple, spaced apart nozzles.

In embodiments, the body 405 of the crimping accessory 400 may include at least one hinge similar to hinge 240 described above such that the crimping accessory 400 has a clam-shell configuration. The clam-shell configuration would allow the crimping accessory 400 to snap onto the shaft 263 of the balloon catheter of the delivery system 260. In other embodiments, the crimping accessory 400 may slide onto the shaft 263 of the balloon catheter of the delivery system 260 via the channel 430.

Similar to the configuration shown in FIG. 2A, once the transcatheter heart valve prosthesis 125 is loaded onto the balloon 261 of the balloon catheter in an expanded configuration and is placed within the crimper chamber 255 of the crimper 250, the crimping accessory 400 slides or snaps onto the shaft 263 of the delivery system 260 and is spaced a distance away from the first end 251 of the crimper 250 such that the crimping accessory 400 may apply pressurized fluid to the at least one leaflet 128 of the transcatheter heart valve prosthesis 125 during the crimping process without directly contacting the transcatheter heart valve prosthesis 125. When the transcatheter heart valve prosthesis 125 is fully crimped onto the delivery system 260, the crimping accessory 400 may be removed from the shaft 263 of the delivery system 260 and the assembly of the transcatheter heart valve prosthesis 125 and the delivery system 260 may be removed from the crimper chamber 255 of the crimper 250.

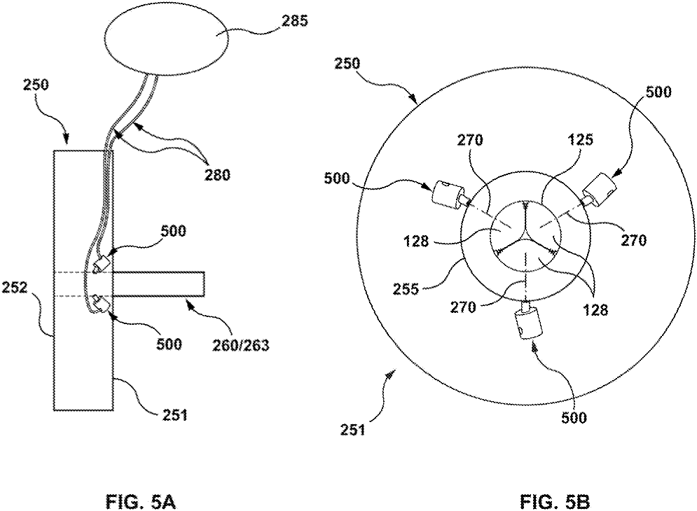

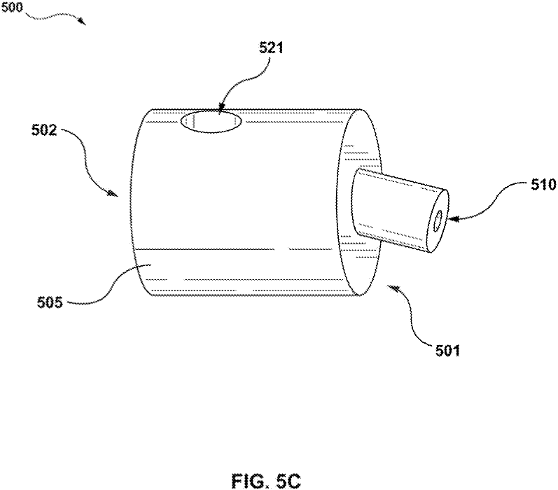

FIGS. 5A-5C show a plurality of crimping accessories 500 according to another embodiment hereof. As compared to previously described embodiments, each crimping accessory 500 described herein is configured to be disposed within the crimper 250 and applies pressurized fluid towards the leaflets 128 of the transcatheter heart valve prosthesis 125 during the crimping process. FIG. 5A shows a schematic side view of the crimper 250, the plurality of crimping accessories 500 disposed within the crimper 250, and a balloon catheter of the delivery system 260 disposed through the crimper chamber of the crimper 250. Similar to previous embodiments described herein, an aperture 521 of each crimping accessory 500 receives a tubing 280 that connects to a fluid source 285 such that the fluid source 285 may provide pressurized fluid to the crimping accessories 500. Each crimping accessory 500 is attached or fixedly secured to an inner surface of the crimper 250, and is oriented and configured to apply pressurized fluid at an angle through the crimper chamber 255 of the crimper 250, towards at least one leaflet 128 of the transcatheter heart valve prosthesis 125 during the crimping process without directly contacting the transcatheter heart valve prosthesis 125.

More particularly, each crimping accessory 500 is attached to an inner surface of the crimper 250 at the first end 250 of the crimper 250 and is positioned such that a nozzle 510 extends into the crimper chamber 255. The nozzle 510 is oriented at an angle to apply or push fluid from the opening at the first end 251 of the crimper 250 towards the leaflets 128 of the transcatheter heart valve prosthesis 125 during the crimping process. In an embodiment, the fluid is applied at an angle such that it travels axially towards the second end 252 of the crimper 250 through the crimper chamber 255 and radially inwards to be directed towards the leaflets 128 of the transcatheter heart valve prosthesis 125 during the crimping process. In an embodiment, the orientation and/or position of the crimping accessory 500 may be adjustable such that the accessory moves radially inward during crimping and the fluid continues to contact the leaflets 128 as the transcatheter heart valve prosthesis 125 is crimped to a smaller diameter. In an embodiment, the crimper 250 is an iris-style crimper that includes a plurality of crimper elements which define at least a portion of the crimper chamber 255, with each of the crimper elements including a surface that forms a portion of the crimper chamber 255. Fluid is directed from the nozzle 510 towards the leaflets 128 of the transcatheter heart valve prosthesis 125 without hitting or interfering with the operation of the crimper elements that form at least a portion of the crimper chamber 255.

FIG. 5B shows a schematic front view of an interior of the crimper 250 to illustrate the relative circumferential positioning of the plurality of crimping accessories 500 disposed within the crimper 250. In this embodiment, more than one crimping accessory 500 is used to apply fluid radially to the at least one leaflet 128 of the transcatheter heart valve prosthesis 125 during the crimping process. In the embodiment shown in FIG. 5B, the valve component 127 of the transcatheter heart valve prosthesis 125 includes exactly three leaflets 128 and exactly three crimping accessories 500 are used to apply fluid to the three leaflets 128 of the transcatheter heart valve prosthesis 125, with each crimping accessory 500 being positioned and oriented so as to apply fluid to a single leaflet of the exactly three leaflets 128. The number of crimping accessories 500 being used may vary from that shown in FIGS. 5A-5C. While the number of crimping accessories 500 preferably correspond with the number of leaflets 128 included in the transcatheter heart valve prosthesis 125, it is not required that the number of crimping accessories 500 match the number of leaflets 128. The assembly may include a greater or lesser number of crimping accessories 500 than the number of leaflets 128.

FIG. 5C shows a perspective view of a single crimping accessory 500 removed from the crimper for sake of illustration only. The crimping accessory 500 includes a body 505, a first end 501, and a second end 502 that opposes the first end 501. The crimping accessory 500 further includes at least one nozzle 510, at least one fluid chamber (not shown), and an aperture 521. The fluid chamber fluidly connects the at least one nozzle 510 to the aperture 521 disposed on an outer surface of the body 505. The aperture 521 is configured to receive the tubing 280 (shown in FIG. 5A), and the tubing 280 is configured to provide fluid from the fluid source 285 to the at least one nozzle 510.

In the embodiment shown, the body 505 contains exactly one nozzle 510 configured to apply pressurized fluid to exactly one leaflet 128 of the transcatheter heart valve prosthesis 125. As described above with respect to FIG. 5B, the valve component 127 of the transcatheter heart valve prosthesis 125 includes exactly three leaflets 128 and each crimping accessory 500 is used to apply fluid radially to a single leaflet of the three leaflets 128 of the transcatheter heart valve prosthesis 125. Each nozzle 510 of each crimping accessory 500 is oriented towards the crimper chamber of the crimper 250 and towards the leaflets 128 of the transcatheter heart valve prosthesis 125 during the crimping process. In addition, each nozzle 510 may be oriented such that fluid applied therefrom is aligned with a center of each leaflet 128. In this embodiment, the three crimping accessories 500 are equally and circumferentially spaced apart from each other as best shown in FIG. 5B so as to optimize alignment with the three leaflets 128.

A method of using the crimping accessory 500 during the crimping process of the transcatheter heart valve prosthesis 125 will now be described. Referring to FIG. 5A, the transcatheter heart valve prosthesis 125 is first loaded onto the balloon of the balloon catheter of the delivery system 260 in an expanded configuration. The assembly of the transcatheter heart valve prosthesis 125 and the delivery system 260 is then positioned within the crimper chamber 255 of the crimper 250. When the crimper 250 operates to radially compress the transcatheter heart valve prosthesis 125 into a crimped configuration, the crimping accessories 500 apply pressurized fluid radially to the leaflets 128 of the transcatheter heart valve prosthesis 125 to urge the leaflets 128 into a substantially closed state during the crimping process. FIG. 5B shows the fluid path 270 in phantom for illustrative purposes only from each crimping accessory 500 to a corresponding leaflet 128 of the transcatheter heart valve prosthesis 125. The fluid from each nozzle 510 is applied to an exterior surface of each leaflet 128 of the transcatheter heart valve prosthesis 125 such that the fluid pushes the leaflets 128 radially inwards. Displacing the leaflets 128 radially inwards during the crimping process minimizes risk of leaflet protrusion (through the frame 126 of the prosthesis 125) and damage. When the transcatheter heart valve prosthesis 125 is fully crimped onto the delivery system 260, the assembly of the transcatheter heart valve prosthesis 125 and the delivery system 260 may be removed from the crimper chamber 255 of the crimper 250.

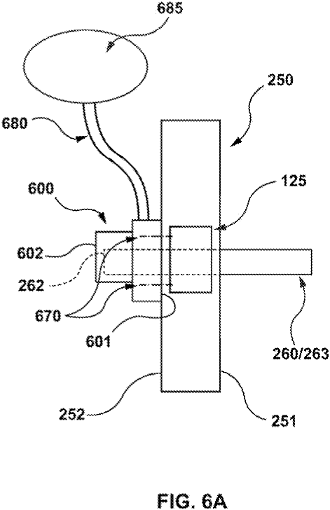

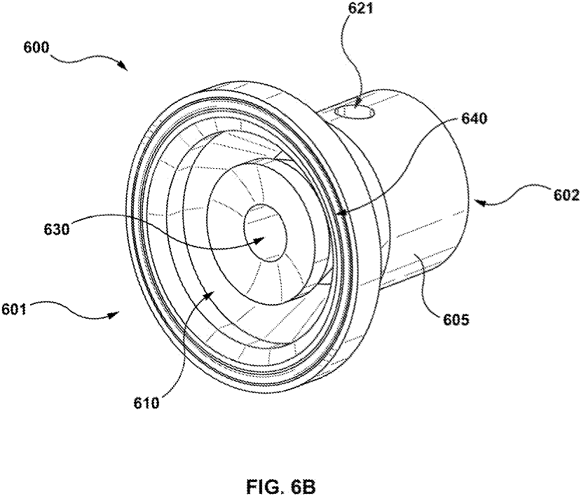

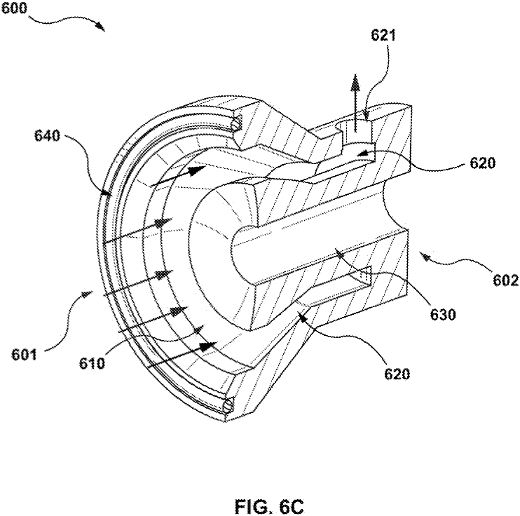

FIGS. 6A-6C show a crimping accessory 600 according to another embodiment hereof. As compared to previously described embodiments, the crimping accessory 600 described herein is configured to suction, or remove, air from the crimper chamber 255 of the crimper 250 such that the vacuum-sealed environment holds the leaflets 128 of the transcatheter heart valve prosthesis 125 in a substantially closed state during the crimping process.

FIG. 6A is a schematic illustration of the crimper 250, the shaft 263 of the delivery system 260, and a crimping accessory 600 during the crimping procedure according to another embodiment hereof. The crimping accessory 600 is configured for use with a transcatheter heart valve prosthesis (such as but not limited to the transcatheter heart valve prosthesis 125 described herein) when radially compressing the transcatheter valve prosthesis into a crimped configuration for delivery within a vasculature. For illustrative purposes only, the crimping accessory 600 will be described for use with the transcatheter heart valve prosthesis 125 since the structure thereof has been described herein. One skilled in the art would understand that when using the crimper 250 to compress a transcatheter valve prosthesis 125, the transcatheter heart valve prosthesis 125 is first loaded onto the balloon of the delivery system 260 in an expanded configuration. The assembly of the transcatheter heart valve prosthesis 125 and the delivery system 260 is then positioned into the crimper chamber 255 of the crimper 250, as can be seen in FIG. 6A. Prior to the crimping procedure, the crimping accessory 600 is positioned over a distal end 262 of the shaft 263 of the delivery system 260 and is securely sealed to an outer surface of the second end 252 of the crimper 250 such that it may remove, or suction, air from the crimper chamber 255 of the crimper 250 during the crimping process without directly contacting the transcatheter heart valve prosthesis 125. Stated another way, the crimping accessory 600 is configured to interact with the leaflets 128 of the transcatheter heart valve prosthesis 125 in a contactless manner. The crimping accessory 600 is connected to a tubing 680 that extends to a suction source 685 such that the suction source 685 may remove air from the crimper chamber 255 through the crimping accessory 600, as will be described in more detail herein.

As shown in FIGS. 6B-6C, the crimping accessory 600 includes a body 605, a first end 601, and a second end 602. The first end 601 and the second end 602 of the crimping accessory 600 are substantially circular in shape. The first end 601 has a first circumference larger than the second circumference of the second end 602. The crimping accessory 600 includes a channel 630 configured to receive the shaft 263 of the balloon catheter of the delivery system 260, as shown in FIGS. 6B and 6C. The channel 630 extends an entire longitudinal length of the crimping accessory 600, extending from the first end 601 to the second end 602 of the body 605. The channel 630 has a substantially circular cross-section and allows the crimping accessory 600 to slide onto the distal end 262 of the shaft 263 of the balloon catheter of the delivery system 260.

The crimping accessory 600 includes at least one nozzle 610, a fluid chamber 620, and an aperture 621, as shown in FIG. 6C. The fluid chamber 620 fluidly connects the at least one nozzle 610 to the aperture 621 disposed on an outer surface of the body 605. In the embodiment shown, the fluid chamber 620 is annular and disposed radially outward from the channel 630 configured to house the shaft 263 of the balloon catheter of the delivery system 260. The aperture 621 is configured to receive the tubing 680, the tubing 680 being configured to connect the crimping accessory 600 to a suction source 685, as can be seen in FIG. 6A. In the embodiment shown, the body 605 contains a singular, annular nozzle 610 configured to remove, or suction, air from the crimper chamber 255 of the crimper 250. The crimping accessory 600 also includes an O-ring or seal 640 on the first end 601 of the body 605. When a suction force is applied via the suction source 685, the applied suction force pulls the crimping accessory 600 into sealing engagement with an outer surface of the crimper 250. The seal 640 is configured to seal the crimping accessory 600 to the outer surface of the second end 252 of the crimper 250 such that the crimping accessory 600 is held airtight to the crimper 250 during the entire crimping process. When the crimping accessory 600 removes the air in the crimper chamber 255 from the second end 252 of the crimper 250, each leaflet 128 is held in a substantially closed state when the crimper 250 is radially compressing the transcatheter heart valve prosthesis 125 into the crimped configuration.

A method of crimping the transcatheter heart valve prosthesis 125 using the crimping accessory 600 is disclosed herein. Referring to FIG. 6A, the transcatheter heart valve prosthesis 125 is first loaded onto the balloon of the delivery system 260 in an expanded configuration. The assembly of the transcatheter heart valve prosthesis 125 and the delivery system 260 is then loaded into the crimper chamber 255 of the crimper 250. The crimping accessory 600 is then disposed over the distal end 262 of the shaft 263 of the delivery system 260. When a suction force is applied via the suction source 685, the applied suction force pulls the crimping accessory 600 onto the outer surface of the crimper 250 such that the crimping accessory 600 is sealed airtight to the outer surface of the second end 252 of the crimper 250. When the crimper 250 operates to radially compress the transcatheter heart valve prosthesis 125 into a crimped configuration, the crimping accessory 600 removes, or suctions, air from the crimper chamber 255 of the crimper 250 during the crimping process without directly contacting the leaflets 128 of the transcatheter heart valve prosthesis 125. The annular nozzle 610 of the crimping accessory 600 allows the removed air to be substantially annular. The arrows in FIG. 6C represent a fluid path 670 within the crimping accessory 600 such that the air is removed from the crimper chamber 255 of the crimper 250 and enters the crimping accessory 600 through the annular nozzle 610. The suction, or removal, of air from the second end 252 of the crimper 250 results in atmospheric pressure pushing against the exterior surfaces of the leaflets 128 and thereby causes the leaflets 128 into a substantially closed state against the shaft 263 of the delivery system 260.

The removal of air from the second end 252 of the crimper chamber 255 of the crimper 250 during the crimping process holds the at least one leaflet 128 in the substantially closed position against the shaft 263 of the delivery system 260 while the crimper 250 radially compresses the transcatheter heart valve prosthesis 125, minimizing the risk of the leaflets 128 protruding through the frame 126 and causing pinching or damage to the leaflets 128. When the transcatheter heart valve prosthesis 125 is fully crimped onto the delivery system 260, the crimping accessory 600 may be removed from the shaft 263 of the delivery system 260 and the second end 252 of the crimper 250, and the assembly of the transcatheter heart valve prosthesis 125 and the delivery system 260 may be removed from the crimper chamber 255 of the crimper 250.

It should be understood that the crimping accessory 600 described herein could be used in conjunction with any embodiments described above such that the crimping accessory 600 vacuums, or removes, the pressurized fluid being applied by the previously mentioned crimping accessories. In such a configuration, a crimping accessory described previously applies pressurized fluid to the leaflets 128 of the transcatheter heart valve prosthesis 125 from the first end 251 of crimper 250, while the crimping accessory 600 described herein removes, or suctions out, the pressurized fluid from the second end 252 of the crimper 250 such that the leaflets 128 are radially displaced into the substantially closed state against the delivery system 260, further minimizing the risk of leaflet protrusion and damage.

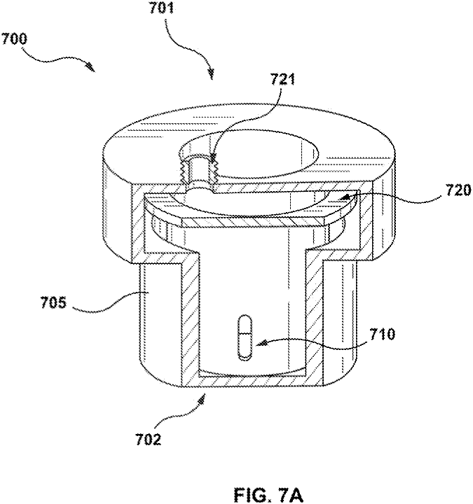

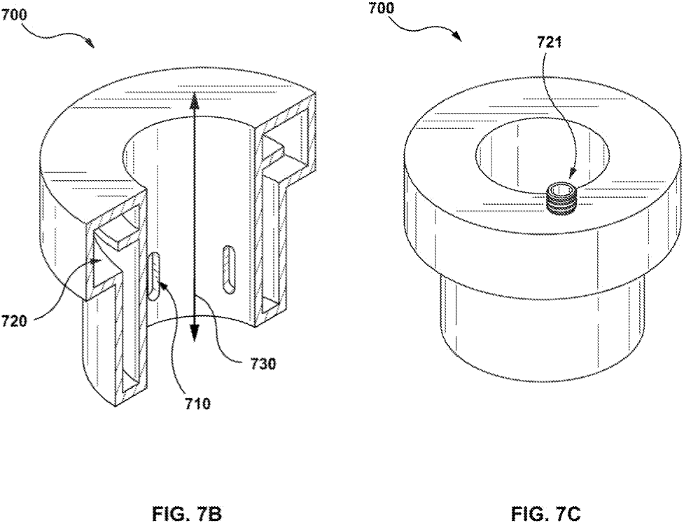

FIGS. 7A-7C show a crimping accessory 700 according to embodiments herein. As compared to the crimping accessories described above, the crimping accessory 700 described herein is configured to be used in between the steps of partially crimping the transcatheter heart valve prosthesis 125 and fully crimping the transcatheter heart valve prosthesis 125. The transcatheter heart valve prosthesis 125 is partially crimped with the crimper 250, then the assembly of the transcatheter heart valve prosthesis 125 and the delivery system 260 is inserted into the crimping accessory 700 such that pressurized fluid is applied radially to the at least one leaflets 128 of the transcatheter heart valve prosthesis 125, and then the assembly is reinserted into the crimper chamber 255 of the crimper 250 such that the transcatheter heart valve prosthesis 125 may be fully crimped onto the delivery system 260, as described in more detail below.

As shown in FIGS. 7A-7C, the crimping accessory 700 includes a body 705, a first end 701, and a second end 702. The first end 701 and the second end 702 of the crimping accessory 700 are substantially circular in shape. The first end 701 has a first circumference larger than a second circumference of the second end 702. The crimping accessory 700 includes a channel 730 configured to receive the transcatheter heart valve prosthesis 125 mounted on the balloon of the delivery system 260. The channel 730 extends an entire longitudinal length of the crimping accessory 700, extending from the first end 701 to the second end 702 of the body 705. The channel 730 has a substantially circular cross-section.

The crimping accessory 700 includes at least one nozzle 710, a fluid chamber 720, and an aperture 721, as shown in FIG. 7A. The fluid chamber 720 fluidly connects the at least one nozzle 710 to the aperture 721 disposed on an outer surface of the body 705. In the embodiment shown, the fluid chamber 720 is annular and disposed radially outward from the channel 730 configured to house the transcatheter heart valve prosthesis 125 on the delivery system 260. The aperture 721 is configured to receive a tubing 280, the tubing 280 being configured to provide fluid from the fluid source 285 to the at least one nozzle 710. In the embodiment shown, the body 705 contains three nozzles 710 configured to apply pressurized fluid radially to the at least one leaflet 128 of the transcatheter heart valve prosthesis 125. In some embodiments, the transcatheter heart valve prosthesis 125 includes exactly three leaflets 128 and the crimping accessory 700 includes exactly three nozzles 710. In such embodiments, each nozzle 710 is configured to apply fluid to a leaflet 128 of the exactly three leaflets 128. Each nozzle 710 is aligned with a center of each leaflet 128 such that when the pressurized fluid is applied, each leaflet 128 is displaced radially inward and may be held in a substantially closed state against the delivery system 260. In this embodiment, the three nozzles 710 are equally and circumferentially spaced apart from each other, as best shown in FIG. 7B.

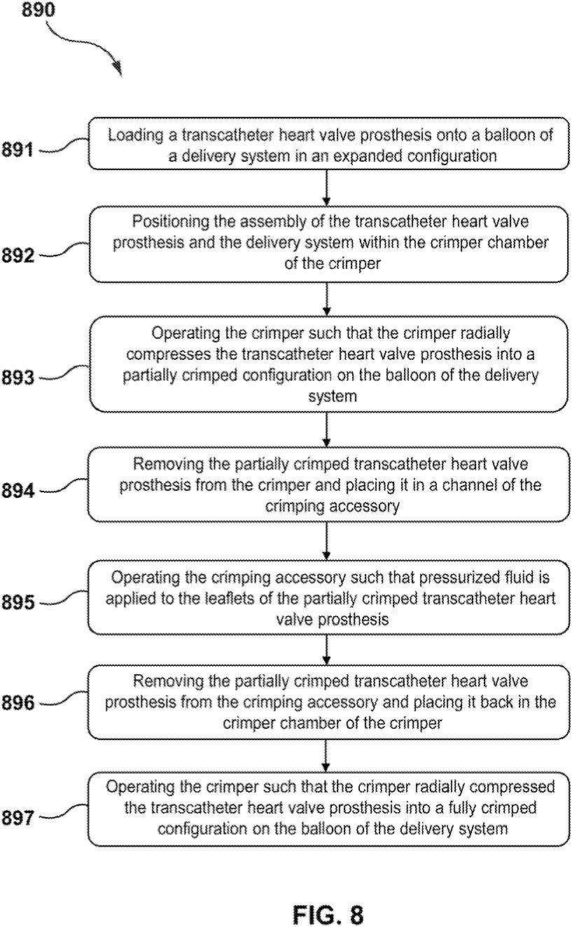

A method of crimping a transcatheter valve prosthesis onto a delivery system utilizing the crimping accessory 700 is disclosed herein. FIG. 8 is a block diagram outlining the steps of a method 890 of utilizing the crimping accessory 700 described herein. In this embodiment, as shown in step 891 and step 892, the transcatheter heart valve prosthesis 125 is loaded onto a balloon of the delivery system 260 in an expanded configuration and positioned within the crimper chamber 255 of the crimper 250. The crimper 250 is operated to radially compress the transcatheter heart valve prosthesis 125 on the balloon of the delivery system 260 into a partially crimped configuration as shown in step 893. The assembly of the partially crimped transcatheter heart valve prosthesis 125 and the delivery system 260 is then removed from the crimper 250 and placed within the channel 730 of the crimping accessory 700 as shown in step 894. When the transcatheter heart valve prosthesis 125 is placed within the channel 730 of the crimping accessory 700, the at least one leaflet 128 of the transcatheter heart valve prosthesis 125 is preferably aligned with the at least one nozzle 710 of the crimping accessory 700. The crimping accessory 700 then applies pressurized fluid radially to the at least one leaflet 128 of the transcatheter heart valve prosthesis 125 such that the at least one leaflet 128 is displaced radially inward and may be held in a substantially closed state against the balloon catheter of the delivery system 260 as shown in step 895. The assembly of the transcatheter heart valve prosthesis 125 and the delivery system 260 is then removed from the crimping accessory 700 and placed back into the crimper chamber 255 of the crimper 250 as shown in step 896. The crimper 250 is then operated to further radially compress the transcatheter heart valve prosthesis 125 into a fully crimped configuration on the delivery system 260 as shown in step 897. The fully crimped transcatheter heart valve prosthesis 125 on the balloon catheter of the delivery system 260 may then be removed from the crimper 250 and is prepared for delivery within a vasculature.

It should be understood that the crimper 250 described herein is merely exemplary in nature and is not intended to limit the invention or the application and uses of the invention. It is understood that any number of alternate crimpers can be used with the leaflet accessory tools and methods described herein. Other non-limiting examples of crimpers that can be used with the leaflet accessory tools described herein are described in U.S. App. No. 17/394,025, filed Aug. 4, 2021, which is incorporated by reference herein in its entirety. In an embodiment, the crimper 250 is an iris-style crimper that includes a plurality of crimper elements which define the crimper chamber 255, with each of the crimper elements including a surface that forms a portion of the crimper chamber 255. A volume of the crimper chamber 255 is decreased in order to radially compress the transcatheter heart valve prosthesis 125 into a crimped configuration on the delivery system 260.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

What is claimed is:

1. An assembly comprising:
a transcatheter heart valve prosthesis including a frame and a valve component including at least one leaflet disposed within and secured to the frame, the transcatheter heart valve prosthesis having a crimped configuration for delivery within a vasculature and an expanded configuration for deployment within a native heart valve; and
a crimping accessory configured for use with a crimper when radially compressing the transcatheter heart valve prosthesis into the crimped configuration, wherein the crimping accessory includes a body having an aperture formed on an outer surface thereof, at least one nozzle coupled to the body, and at least one fluid chamber formed between the aperture and the nozzle such that the aperture and the at least one nozzle are in fluid communication, the crimping accessory being configured to apply or remove pressurized fluid via the at least one nozzle to or from the at least one leaflet of the transcatheter heart valve prosthesis to hold the at least one leaflet in a substantially closed state when the crimper is radially compressing the transcatheter heart valve prosthesis into the crimped configuration.

2. The assembly of claim 1, wherein the crimping accessory includes a first end and a second end opposing the first end, the first end and the second end of the crimping accessory being substantially circular in shape.

3. The assembly of claim 2, wherein the first end of the crimping accessory has a first circumference which is larger than a second circumference of the second end of the crimping accessory.

4. The assembly of claim 1, wherein the aperture disposed on the outer surface of the body is configured to receive tubing configured to connect the crimping accessory to a fluid source or a suction source.

5. The assembly of claim 1, wherein the body of the crimping accessory further includes a channel extending from a first end to a second end thereof, the channel being configured to receive a balloon catheter of a delivery system for the transcatheter heart valve prosthesis.

6. The assembly of claim 5, wherein the body of the crimping accessory further includes at least one hinge such that the crimping accessory has a clam-shell configuration that allows the crimping accessory to snap onto the balloon catheter of the delivery system.

7. The assembly of claim 1, wherein the fluid chamber is annular and is disposed radially outward from the channel configured to receive the balloon catheter.

8. The assembly of claim 1, wherein the transcatheter heart valve prosthesis includes exactly three leaflets and the crimping accessory includes exactly three nozzles, each nozzle being configured to remove or apply fluid to or from a leaflet of the exactly three leaflets.

9. The assembly of claim 1, wherein the body of the crimping accessory includes a slot extending from a first end to a second end thereof, the slot being configured to receive a balloon catheter of a delivery system for the transcatheter heart valve prosthesis, and wherein the crimping accessory slides onto the balloon catheter of the delivery system via the slot.

10. The assembly of claim 9, wherein the slot configured to house the balloon catheter extends an entire longitudinal length of the crimping accessory and has a substantially U-shaped cross-section, and wherein the fluid chamber is C-shaped and disposed radially outward from the slot configured to house the balloon catheter.

11. The assembly of claim 1, wherein the crimping accessory is configured to be disposed within and attached to an interior surface of the crimper.

12. The assembly of claim 1, wherein the crimping accessory contains a singular, annular nozzle that at least one of applies pressurized fluid to and removes pressurized fluid from the at least one leaflet of the transcatheter heart valve prosthesis.

13. The assembly of claim 12, wherein the crimping accessory includes a seal around an outer perimeter of the first end configured to seal around the crimper.

14. A method of crimping a transcatheter heart valve prosthesis onto a delivery system, comprising:
positioning a transcatheter heart valve prosthesis into a crimper chamber of a crimper, the transcatheter heart valve prosthesis including a frame and a valve component including at least one leaflet disposed within and secured to the frame, wherein the transcatheter heart valve prosthesis is in an expanded configuration;
operating the crimper to radially compress the transcatheter heart valve prosthesis into a crimped configuration for delivery within a vasculature, wherein a crimping accessory applies or removes pressurized fluid to or from the at least one leaflet of the transcatheter heart valve prosthesis to hold the at least one leaflet in a substantially closed state during the step of operating the crimper.

15. The method of claim 14, wherein the transcatheter heart valve prosthesis may be partially compressed or fully compressed during the operating step.

16. The method of claim 14, wherein the crimping accessory includes a body, at least one nozzle and at least one fluid chamber fluidly connecting the at least one nozzle to an aperture disposed on an outer surface of the body, the at least one nozzle being configured to apply or remove pressurized fluid to or from the at least one leaflet of the transcatheter heart valve prosthesis to hold the at least one leaflet in a substantially closed state when the crimper is radially compressing the transcatheter heart valve prosthesis into the crimped configuration.

17. The method of claim 14, wherein the crimping accessory is positioned outside the crimper.

18. The method of claim 14, wherein the crimping accessory is positioned within the crimper.

19. A method of crimping a transcatheter heart valve prosthesis onto a delivery system, comprising:
positioning a transcatheter heart valve prosthesis into a crimper chamber of a crimper, the transcatheter valve prosthesis including a frame and a valve component including at least one leaflet disposed within and secured to the frame, wherein the transcatheter heart valve prosthesis is in an expanded configuration and is mounted over a balloon of a balloon catheter;
operating the crimper to radially compress the transcatheter heart valve prosthesis on the balloon of the balloon catheter into a partially crimped configuration;
removing the partially crimped transcatheter heart valve prosthesis on the balloon catheter from the crimper and placing it within a crimping accessory;
while disposed within the crimping accessory, applying pressurized fluid to the at least one leaflet of the transcatheter heart valve prosthesis to hold the at least one leaflet in a substantially closed state;
removing the partially crimped transcatheter heart valve prosthesis on the balloon catheter from the crimping accessory and placing it back in the crimper chamber of the crimper;
operating the crimper to further radially compress the transcatheter heart valve prothesis into a fully crimped configuration on the balloon catheter configured for delivery within a vasculature; and
removing the transcatheter heart valve prosthesis in the crimped configuration on the balloon catheter of the delivery system from the crimper.

20. The method of claim 19, wherein the crimping accessory includes a body, at least one nozzle and at least one fluid chamber fluidly connecting the at least one nozzle to an aperture disposed on an outer surface of the body, the at least one nozzle being configured to radially apply pressurized fluid to the at least one leaflet of the transcatheter heart valve prosthesis to hold the at least one leaflet in the substantially closed state when the crimper is radially compressing the transcatheter heart valve prosthesis into the crimped configuration.
